(19) United States
(12) Patent Application Publication
(10) Pub. No.: US 2023/0166251 A1
(43) Pub. Date: Jun. 1, 2023

(54) ALL-IN-ONE MICROCHAMBER FOR 3D MUSCULAR TISSUES

(71) Applicant: Biond Solutions B.V., Delft (NL)

(72) Inventors: Nikolas Gaio, Delft (NL); William Fausto Quiros Solano, Delft (NL); Amr Abdelhameed Mohamed Othman, Delft (NL); Cinzia Silvestri, Delft (NL)

(21) Appl. No.: 17/974,891

(22) Filed: Oct. 27, 2022

Related U.S. Application Data
(63) Continuation of application No. PCT/NL2021/050201, filed on Mar. 25, 2021.

(30) Foreign Application Priority Data
Apr. 28, 2020 (NL) 2025441

(51) Int. Cl. B01L 3/00

(52) U.S. Cl. CPC B01L 3/502715; B01L 2300/0645; B01L 2300/0861; B01L 2300/0848; B01L 2400/0638; B01L 2300/0609; B01L 2200/10; B01L 2300/0663; B01L 2300/1827; B01L 2300/0816

(57) ABSTRACT

The present invention is in the field of an all-in-one microchamber for 3D muscular tissues, wherein at least one 3D microenvironment is present, a method of producing said device using silicon-based technology, and a use of said device in various applications, typically a biological cell experiment, such as a cell or organ-on-a-chip experiment, and lab-on-a-chip experiment, and use of the device as a micro-reactor.

### CROSS-REFERENCE TO RELATED APPLICATIONS

This application is a continuation of International Patent Application No. PCT/NL2021/050201, titled “ALL-IN-ONE MICROCHAMBER FOR 3D MUSCULAR TISSUES”, filed on Mar. 25, 2021, which claims priority to and the benefit of Netherlands Patent Application No. 2025441, titled “ALL-IN-ONE MICROCHAMBER FOR 3D MUSCULAR TISSUES”, filed on Apr. 28, 2020, and the specification and claims thereof are incorporated herein by reference.

### BACKGROUND OF THE INVENTION

### Field of the Invention

The present invention is in the field of an all-in-one microchamber for 3D muscular tissues, wherein at least one 3D microenvironment is present, a method of producing said device using silicon-based technology, and a use of said device in various applications, typically a biological cell experiment, such as a cell or organ-on-a-chip experiment, and lab-on-a-chip experiment, and use of the device as a micro-reactor.

A microfluidic device relates to a set of technologies with an aim to manipulate at least one small fluid (liquid or gas) volume within microsystems produced by human beings. In the device a cell culture or an individual cell or the like may be present. An experiment on said cell culture refers to the maintenance and growth of cells in a well-controlled environment. The environment may resemble naturally occurring circumstances. As such a cell can likewise be studied under application of at least one of numerous signals that might be present in its naturally occurring surrounding microenvironment.

A microfluidic cell culture may attempt to manipulate cells, such as by culturing, maintaining, and growing, and qualitatively and quantitatively experimenting and analyzing cells in microfluidic volumes. Such may relate to an attempt to understand a cell culture, such as a stem cell culture, non-dividing or slowly dividing cells, e.g. in terms of an interaction between cell culture parameters and the micro environmental conditions created by microfluidic devices. It is considered that dimensions of the microfluidics, such as chamber and channels, are well suited to the physical scale of the biological cells and other applications.

In general, it is considered that microfluidics provides a good degree control over e.g. cell culture conditions. Typically a movement of fluids in the microfluidics is considered to be laminar; a fluid volume is typically in the order of 10⁻⁶-10⁻¹² l, which volume may flow in 10-60 seconds; fluid flow may be controlled precisely in terms of volume and timing, such as by providing an in-chip valve; also precise chemical and physical control of the microenvironment is possible; a production of a multitude of individually controllable cell culture chambers on a single device is considered, albeit typical prior art technologies rely on manual procedures which are considered to be insufficiently controlled.

### Background Art

Some prior art documents recite microfluidic devices. WO2016/049363 A1, WO2016/049365 A1, WO2016/010861 A1, WO2016/004394 A1, and US15/2955534 A1 recite relatively simple organ-on-chip devices, which cannot include any complex sensing/stimulation elements; hence these devices are not unsuited for most applications.

The present inventors have filed an international application, WO 2018/021906 A1, comprising basic concepts of such microfluidic devices.

### BRIEF SUMMARY OF THE INVENTION

Further developments have been aimed at certain aspects of microchambers with advanced functionality, but typically at one aspect at a time. For instance, supports for a gel matrix can be provided. Or a reaction chamber with advanced microfluidic channels providing flow paths is provided, aimed at trapping cells or the like. Or cantilevers for supporting cells. Or media for supporting cell maturation.

The present invention relates to a device and a method of producing a versatile and advanced device which overcomes one or more of the above or further disadvantages, without jeopardizing functionality and advantages.

The present invention relates in a first aspect to a micro-fluidic device 100 comprising at least one first microchamber 10a having a bottom 11 and at least one wall 12, preferably wherein the first microchamber comprises an opening such that it is directly accessible from outside, in the microchamber at least one pillar 20 extending from the bottom 11 upwards for supporting of cell or tissue, i.e. pillars are resting on the bottom, preferably wherein the at least one pillar has a height of 1-2000 μm, and preferably wherein the at least one pillar has a width of 1-2000 μm, and at least one first channel 30 in fluidic contact with the at least one microchamber 10a embedded in the bottom 11, wherein the bottom 11 comprises a porous membrane 13 so as to embody a selective barrier providing fluidic contact between the at least one first channel 30 and the at least one microchamber 10a. The porous membranes allows to selectively isolate the microfluidic channel, allowing to inject cells inside the channel (without having them enter in the microchamber), and cover the four walls of the channel, creating a structure that resembles a blood vessel very close to the tissue in the microchamber. The pores in the membrane can be designed so that some cells do not pass through them, while nutrients do. Moreover, the pores can be designed to allow for angiogenesis and vascularization starting from the microfluidic channel.

The device according to claim 1 has further advantages of a higher throughput, being cheaper to produce, being more reliable and more versatile, providing a better handling of e.g. cells, and providing a much wider functionality.

First and second microchambers typically have a dimension (e.g. cross-section) in the order of 5-1000 μm, preferably 10-500 μm such as 100-300 μm. First and second microchannels typically have a dimension (e.g. cross-section) in the order of 50-1000 μm, preferably 100-700 μm such as 400-500 μm.

The present device comprises at least two distinct layers in which microfluidic and nano-/microscale elements and the like are provided. Two layers are typically made of a polymer, typically but not necessarily the same polymer for both layers; a first polymer layer 13a is provided on a substrate, typically silicon or a glass wafer, and is relatively thin; for the purpose of the invention the terms “substrate”, “silicon”, and “glass” are considered interchangeable; the top layer may be considered to relate to a membrane, which is considered to relate to a selective barrier; the top layer preferably is provided with a matrix of openings (or holes) 11a therein, the at least one hole allowing passage of e.g. fluids, gases, species, micro-particles, ions, etc. which can be adapted for specific uses; the top layer has a thickness of 0.05-30 μm, preferably 0.1-25 μm, more preferably 0.2-20 μm, even more preferably 0.5-10 μm thin, such as 1-8 μm or 2-6 μm; in contact with the relative thin polymer layer 13a is a thicker polymer bottom layer 13b; the bottom layer may comprise at least one second micro-channel 31 and/or at least one second micro-chamber 10b at least partly embedded in the polymer bottom layer; the number, layout, sizes, and further characteristics of these microfluidics can be adapted for specific uses; the microfluidics may be embedded fully in the bottom layer 13b and/or may be embedded partly, such as in the case of a well; the polymer bottom layer is thicker than the top layer and preferably has a thickness of 50-2000 μm, hence is at least one order of magnitude thicker than the top layer, and typically 2-3 orders of magnitude thicker; the thickness is preferably 150-1000 μm, more preferably 200-500 μm, even more preferably 250-400 μm; the device further comprises silicon based microfluidics in microfluidic contact with the top layer 13a of the polymer based microfluidics wherein the silicon based microfluidics are accessible and/or can be made accessible for use of the device; the substrate, e.g. silicon, based microfluidics comprise at least one first micro-channel 30 and/or at least one first micro-chamber 10a at least partly embedded (see above) in the silicon, and may comprise at least one input 16, wherein the input 16 is in microfluidic contact with the at least one second micro-channel 31 and/or at least one second micro-chamber 10b embedded in the polymer bottom layer, e.g. as functionally defined or required; the support or substrate 10 may relate to a typically used wafer in a silicon semiconductor process such as of Si or glass; wherever silicon is mentioned in this respect it may relate to any other suitable substrate; the polymer top layer 13a is for separating (fluidics in the) at least one of the first micro-channel 30 and/or at least one of the first micro-chamber 10a embedded in the substrate (silicon) from (fluidics in the) at least one of the second micro-channel 31 and/or at least one of the second micro-chamber 10b embedded in the polymer bottom layer preferably at least partly by the matrix of holes 11a therein; the microfluidics of the polymer and silicon are directly or indirectly in microfluidic contact with one and another. The present polymer is independently selected from biocompatible polymers, such as polysiloxanes, such as polydimethylsiloxane (PDMS), polyimides, polyurethane, styrene-ethylene-butylene-styrene (SEBS), polypropylene, polycarbonate, polyester, polypropylene, and butyl rubber, and from biodegradable polymers, such as Biorubber (poly(glycerol sabacate PGS), and poly(1,8-octanediol-co-citrate) (POC), and combinations thereof.

The term “fluidics” may relate to a gas, a liquid; and combinations thereof; a “microfluidic” is considered to relate to a fluid under boundary conditions of the device.

The set-up composed by the polymer layers, typically forming a polymer film, the at least one micro-channel, such as 2-10 micro-channels, the at least one micro-chamber, such as 2-10 microchambers, and first micro-chamber (also referred to as “macro-chamber”) can be optically monitored off-line with a microscope and/or a camera e.g. placed on a backside/front-side of the device. In many of the examples only one macro-chamber 10a is present. The set-up can be monitored on-line by means of micro-electrode array and/or micro-fabricated sensors (such as flow/temperature/pH sensor) placed in the micro-environment and or in the macro-chambers. The set-up can be also altered/stimulated by means of liquid flow flowing through the micro-chamber/channels and the macro-chamber; likewise by gas flow flowing through the micro-chamber/channel and the macro-chamber; by pressure differences applied in the micro-chambers, in the micro-channels and on the backside and the front side of the polymer film; by electrical stimulation provided by means of microelectrode arrays; by optical stimulation provided with optical systems placed on the backside/front-side of the device; by chemical stimulation provided by means of liquid flow or liquid reservoir placed in the membrane; and other micro-fabricated actuators placed inside the micro-channel/chambers; and combinations thereof, hence the device is considered to be versatile.

In a second aspect the present invention relates to a method of applying a stimulus to at least one living organism or living part thereof, comprising providing at least one micro-fluidic device (100) according to the invention, providing the at least one living organism or living part thereof each individually comprising at least one cell, such as in microchamber 10a or in channel 30, providing at least one stimulus to the at least one living organism or living part thereof, and obtaining a test result.

The terms “at least one living organism or living part thereof” are considered to relate to actual organisms, parts thereof, such as cell lines, tissue, etc., biological material resembling living organisms or parts thereof, and any other similar form of biological material, such as identified throughout the application and claims.

Thereby the present invention provides a solution to one or more of the above-mentioned problems.

Advantages of the present invention are detailed throughout the description.

### BRIEF DESCRIPTION OF THE SEVERAL VIEWS OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more embodiments of the invention and are not to be construed as limiting the invention. In the drawings:

FIGS. 1a, 1b, 1c, and 1d show details of an exemplary embodiment of one device that includes two pillars, one channel accessible through inlet and an outlet and through hole matrix in the channel. FIG. 1a: side view. FIG. 1b: top view.

FIGS. 2a, 2b, and 2c show details of an exemplary embodiment of one device with oval well, equipped with two pillars and a microfluidic channel. FIG. 2a shows details of an exemplary embodiment of one device with pointer that can be used as optical guiders to identify the displacement of the pillars caused by the contraction of the muscle bundle. FIG. 2c shows a close up of an array of openings present in the device.

FIGS. 3a, 3b, 3c, 3d, 3e, 3f, 3g and 3h show details of the microfluidic channel inside the chip. FIGS. 3b-c are cross-sections of FIG. 3a, and FIGS. 3e-f are cross-sections of FIG. 3d.

FIGS. 4a, 4b, 4c, and 4d show images of details of the present device with (FIGS. 4a,b) and without (FIGS. 4c,d) a muscle bundle supported by the two pillars present in the device. The arrow indicates a flow path.

FIGS. 5a, 5b, 5c, 5d, 5e, and 5f show cross-sectional details of the present invention. FIGS. 5a-f show details of an exemplary embodiment of one device when it is in relaxed state with and without muscle bundle, when the polymer layers are stretched by applying a difference of pressure between the microchamber and the back of the thick polymer layer. FIG. 5e shows details of an exemplary embodiment of one device with a pillars electrically accessible via contact pads, to stimulate and monitor the tissue and cells inserted in the chip. The figure reflects a micro-fluidic device further comprising at least one electrode 41 in the bottom, wherein the electrode is preferably provided in the bottom top layer 11a, wherein an electrode at one end is in electrical contact with at least one pillar 20, and wherein the electrode comprises a contact 41a at another end thereof, such as a pad, wherein optionally the electrode is incorporated in an insulating material 41b, and wherein optionally the contact 41a is electrically separated from the wall 12 by a further insulating material 41c. In FIG. 5e contact pad 41a, and pillar 20 being electrically accessible via contact pad 41a, are shown.

FIGS. 6a and 6b show details of an exemplary embodiment of one device wherein the cross-section of the pillars gradually increases in area from the bottom upwards. FIGS. 6c and 6d show details of an exemplary embodiment of one device with pointer that can be used as optical guiders to identify the displacement of the pillars caused by the contraction of the muscle bundle.

FIG. 7 shows a further cross-section of an exemplary embodiment of one device wherein at least one pillar 20 is hollow, preferably wherein the hollow part of the pillar 20 is in microfluidic connection with first channel 30, or with second channel 31, or with first or second microchamber 10a, 10b. The arrow indicates a flow path.

FIG. 8 shows a device comprising a support 60, such as a plate, wherein the micro-fluidic device is preferably detachably attached to said support 60.

FIGS. 9a, 9b, 9c, and 9d show a further cross-section of an exemplary embodiment of one device without (a) and with (b, c, d) cells cultured inside the device. The pillars 20 can be employed to support a muscle bundle 50 (FIG. 9b). Cells can be also cultured inside the microfluidic channel (FIG. 9c) or on the bottom of the macro chamber (FIG. 9d). Cell cultures shown in FIGS. 9b, 9c, 9d can be combined.

FIGS. 10a, 10b, and 10c depict several possible shapes of the microchamber of the device of the invention.

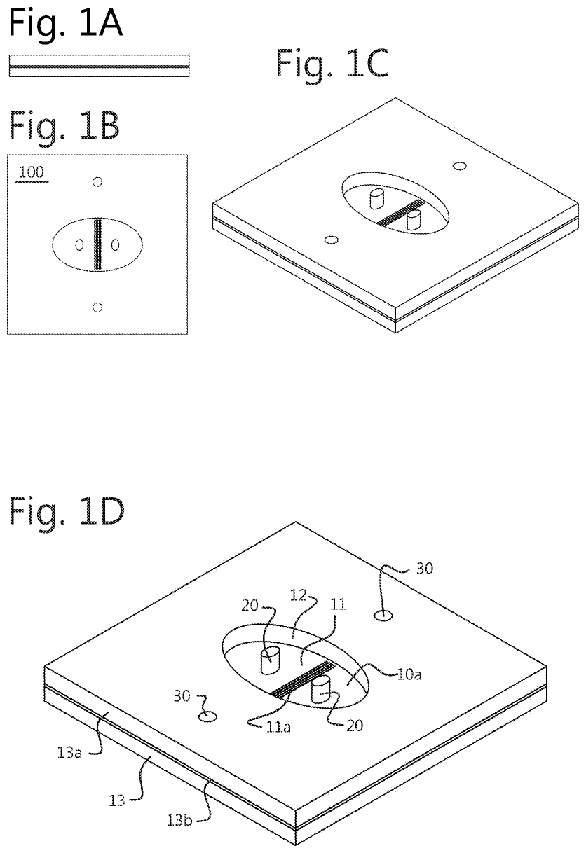

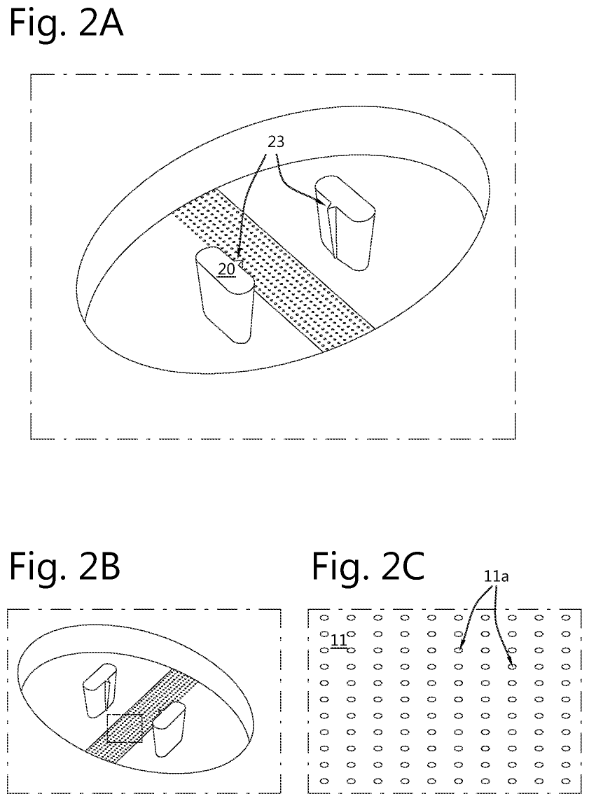

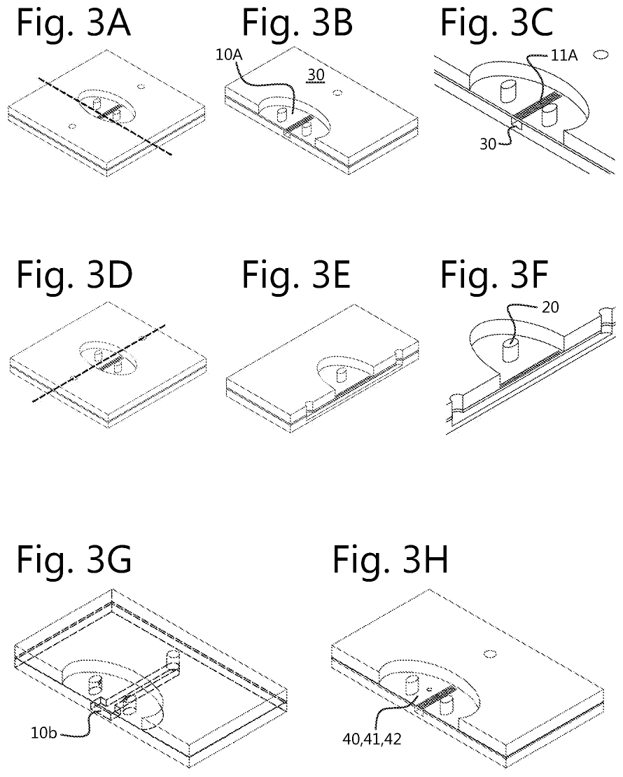

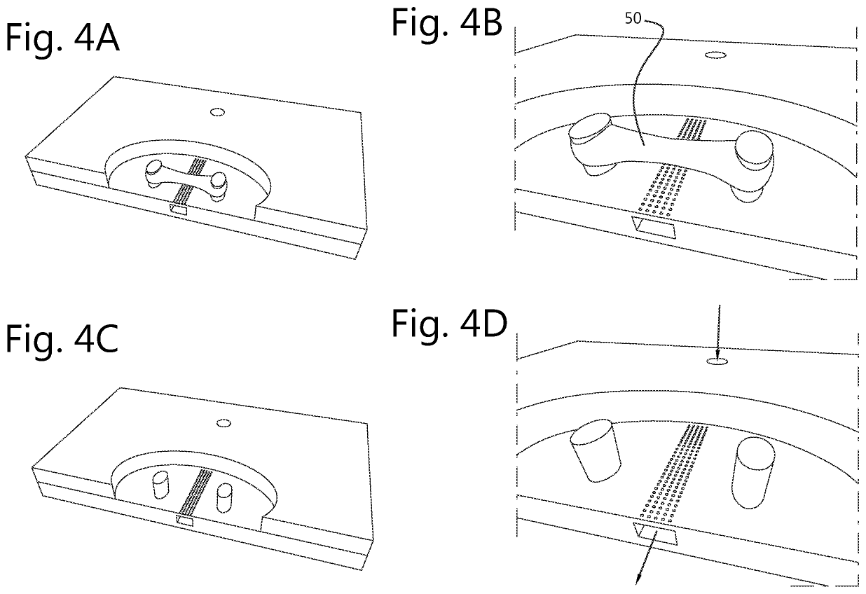

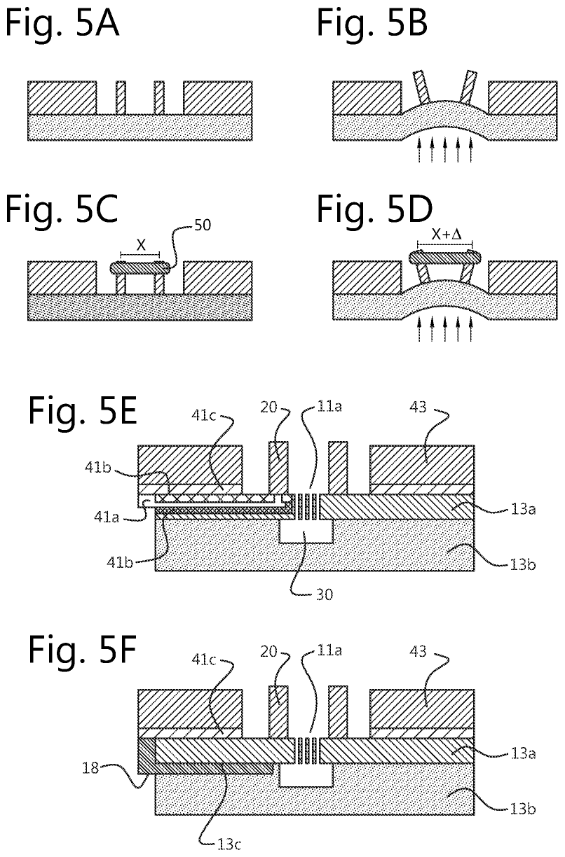

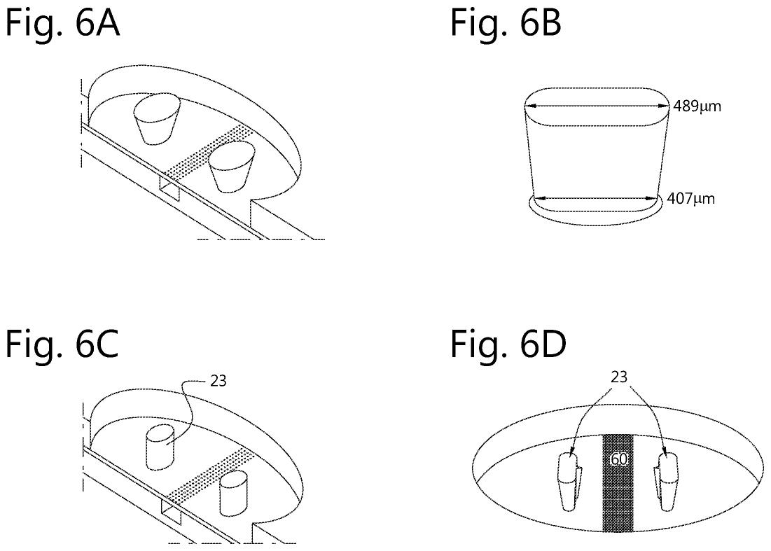

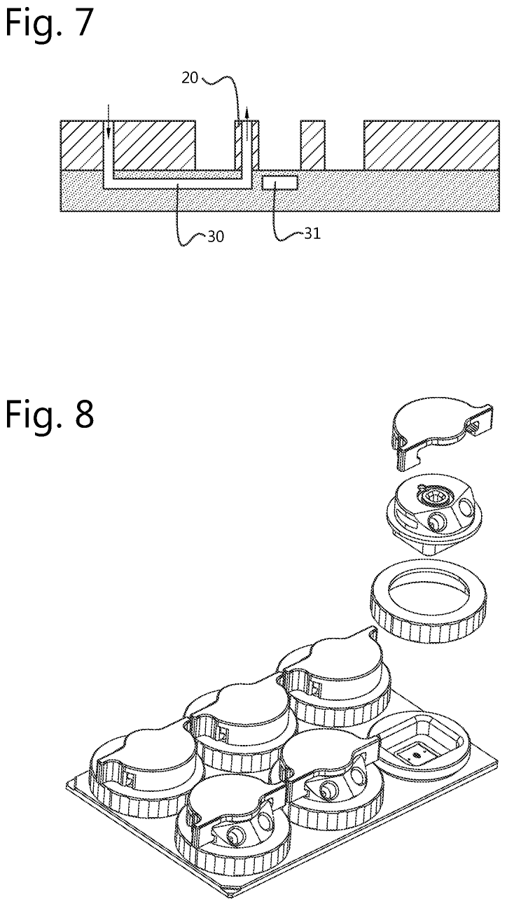

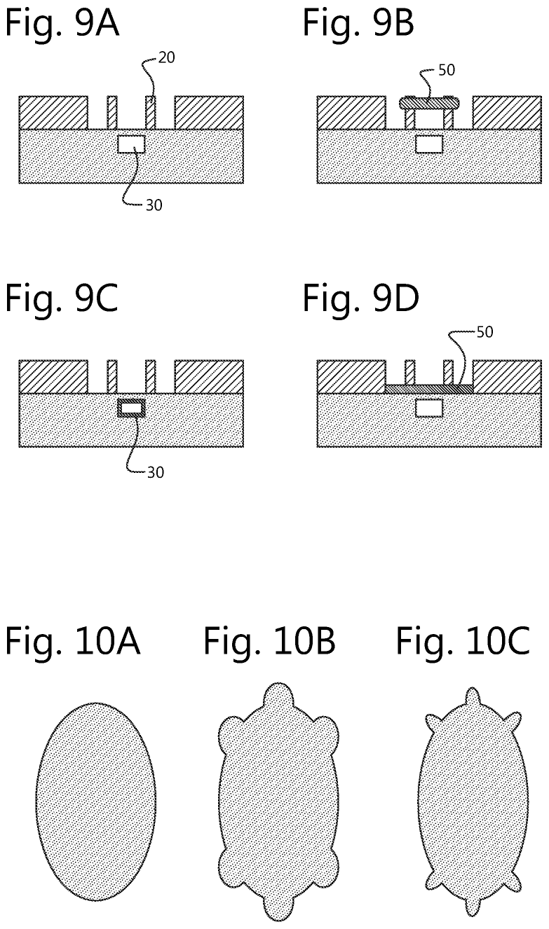

### DETAILED DESCRIPTION OF THE FIGURES

In the figures the numbers below depict the features that are mentioned thereafter:
100 Micro-fluidic Device
10a first microchamber
10b second microchamber
11 bottom of first microchamber
11a opening
12 wall of first microchamber
13 polymer film
13a thin polymer top layer
13b polymer bottom layer
13c metal bottom layer
18 output
20 pillar
23 optical guider
30 first microchannel
31 second microchannel
40 stimulator
41 electrical stimulator
41a electrode contact
41b electrode insulator material
41c electrode/wall insulator
42 mechanical stimulator
43 silicon
50 tissue (e.g. muscle bundle)
60 support

### DETAILED DESCRIPTION OF THE INVENTION

The present invention relates in a first aspect to a device according to claim 1.

In an exemplary embodiment the present device may further comprise at least one stimulator 40, such as an electrical stimulator 41, such as an electrode, a chemical stimulator, an optical stimulator, and a mechanical stimulator 42, such as a pump pneumatical pressure/force, and/or a tissue monitor, such as an electrode. With said stimulator an effect thereof can be monitored or studied on the living organism, or part thereof.

In an exemplary embodiment of the present device the bottom 11 may comprise at least one opening 11a, preferably an array of openings 11a, such as 1-100 openings 11a/μm².

In an exemplary embodiment of the present device the array comprises n×m openings, wherein n>10, and m>10.

In an exemplary embodiment of the present device a density of holes may be 0.001-250/100 μm².

In an exemplary embodiment of the present device an average hole area may be 0.05-500 μm².

In an exemplary embodiment of the present device the at least one pillar may preferably be provided near and/or on top of the at least one opening.

In an exemplary embodiment of the present device the bottom 11 may comprise a polymer film 13, preferably wherein the polymer film comprises a 0.05-100 μm thin polymer top layer 13a the polymer top layer preferably having a matrix of holes 11a therein.

In an exemplary embodiment of the present device the bottom 11 may comprise a 50-5000 μm polymer bottom layer 13b in contact with the polymer top layer, and optionally comprising at least one second micro-channel 31 and/or at least one second micro-chamber 10b at least partly embedded in the polymer bottom layer.

In an exemplary embodiment of the present device the at least one pillar is preferably provided near and/or on top of the at least one first or second micro-chamber.

In an exemplary embodiment of the present device wherein the at least one pillar is preferably provided near and/or on top of the at least one first or second micro-channel.

In an exemplary embodiment the at least one pillar is selectable to be of a rigid construction or of a flexible construction. The advantage of this aspect is that the functionality of the pillars can be increased (for example, by being used as 3D electrodes). Moreover, with pillars that can be both flexible and rigid, the forces experienced by the tissues anchored to the two pillars can be tuned.

In an exemplary embodiment of the present device the polymer may independently be selected from biocompatible polymers, such as poly siloxanes, such as polydimethylsiloxane PDMS, polyimides, polyurethane, butyl rubber, styrene-ethylene-butylene-styrene SEBS, polypropylene, polycarbonate, polyester, polypropylene, and biodegradable polymers, such as Biorubber PGS and poly1,8-octanediol-co-citrate POC, and combinations thereof.

In an exemplary embodiment of the present device the thin polymer top layer 13a may comprise at least one side thereof, at least one micro-feature, such as an indentation, a groove, a topographical structure, preferably at least one oriented microgroove, preferably an array of x*y oriented microgrooves, wherein a density of microgrooves is 1-25/100 μm².

In an exemplary embodiment of the present device an average groove area may be 0.1-106 μm2.

In an exemplary embodiment of the present device the at least one micro-feature may be aligned with respect to the device.

In an exemplary embodiment of the present device the polymer layers 13a, 13b are provided with at least one access, the at least one access providing access to at least one of a metal pad, an IC, a sensor, such as an optical sensor, and a heater.

In an exemplary embodiment of the present device a rigid substrate may form the microchamber wall 12, such as a silicon substrate.

In an exemplary embodiment of the present device the least one first microchamber 10a may have a shape resembling a biological tissue or organism to be received.

In an exemplary embodiment the present device may comprise at least one electrode 41 in the bottom.

In an exemplary embodiment of the present device the electrode is preferably provided in the bottom top layer 11a.

In an exemplary embodiment of the present device the electrode is provided in microchannel 30.

In an exemplary embodiment of the present device the electrode is provided on layer 13a.

In an exemplary embodiment of the present device an electrode at one end may be in electrical contact with at least one pillar 20.

In an exemplary embodiment of the present device the electrode may comprise a contact 41a at another end thereof, such as a pad, wherein optionally the electrode is incorporated in an insulating material 41b, and wherein optionally the contact 41a is electrically separated from the wall 12 by a further insulating material 41c. It is possible to contact the electrode with the contact 41a through an intermediate metal line, which may be straight or meandering or which may follow any other suitable path.

In an exemplary embodiment of the present device the bottom may comprise a metal layer 13c, preferably in between a top layer 13a and bottom layer 13b, wherein the metal layer is preferably patterned, and wherein the metal layer is adapted to detect deformation of the at least one pillar, such as for strain gauges that measure the bending of the pillars to detect the contraction of the cell bundle attached to the pillars.

In an exemplary embodiment of the present device the at least one pillar 20 may have a cross-section selected from square, rectangular, oval, elliptic, circular, triangular, multigonal, and combinations thereof.

The shape of the first microchamber can be tailored to reduce the stress inflicted on the electrodes and the intermediate metal lines. A possible option is for instance to apply the cross-sectional shape shown in FIGS. 10b and 10c, which depicts an essentially elliptical shape provided with three protrusions on the opposite heads of the virtual relatively long axis of the ellipse.

In an exemplary embodiment of the present device a cross-section may be substantially constant from the bottom upwards, or wherein the cross-section gradually may increase in area from the bottom upwards.

In an exemplary embodiment of the present device at least one pillar may be provided with an optical guider 23 at a top thereof, such as a pointer, such as a triangular pointer, wherein optical guiders of opposite pillars may point in the same direction, or in an opposite direction, and combinations thereof.

In an exemplary embodiment of the present device at least one pillar may be hollow, preferably wherein the hollow part of the pillar is in microfluidic connection with channel 30, or with channel 31, or with microchamber 10a or 10b.

In an exemplary embodiment the present device may further comprise embedded in the device at least one of a pump, or adapted to receive fluid from a pump, a valve, a strain gauge, an actuator, a heater, a cooler, a flow sensor, a temperature sensor, a pH sensor, an IC-circuit, an amplifier, an actuator, a hot plate, a micro-electrode array, an ion sensor, a pressure regulator, further microfluidic elements, at least one of a microchip, an integrated sensor, and an output 18, preferably embedded in the bottom 11, such as in a rigid part thereof.

In an exemplary embodiment of the present device a wet/humid section and a dry section of the device may be physically separated, wherein the dry section comprises electronics.

In an exemplary embodiment of the present device the bottom may comprise a polymer film which is stretchable having a tensile strength of >1 [MPa] (ISO 527) and/or flexible with a Young's modulus of <3 [GPa] (ISO 527).

In an exemplary embodiment of the present device the polymer film may be rigid having a Young's modulus of >10 [GPa] (ISO 527).

In an exemplary embodiment the present device may further comprise a support 60, such as a plate, wherein the micro-fluidic device is preferably detachably attached to said support.

In an exemplary embodiment the present device may comprise at least one living organism or living part thereof, wherein the at least one living organism is selected from undifferentiated cells, differentiated cells, mature cells, stem cells, such as adherent or suspension primary cells, transfected or non-transfected cell lines, adult, embryonic or induced pluripotent stem cells, tissues, tissues inserts, and 3D microtissues, such as muscles and cardiac micro tissues, 3D cultures, such as spheroids and organoids, and combinations thereof.

In an exemplary embodiment of the present method the stimulus may be a chemical stimulus, a mechanical stimulus, an electrical stimulus, or an optical stimulus, such as for toxicity testing of drugs and xenobiotics, for efficacy testing, for modeling barrier tissues in vitro, for integrity assessment and drug transport assays, for Drug metabolism studies, for drug pharmacokinetic and toxicokinetic studies, for metabolizing organ and targeting pharmacological organs, for disease modelling, for disease diagnosis, for studying a disease mechanism, for prognosis, for personalized and precise medicine, for doping, for microgravity studies, for drug interaction, for cell maturation, and for cell differentiation.

In an exemplary embodiment of the present method the method is in vitro.

In an exemplary embodiment of the present method the at least one living organism may be selected from undifferentiated cells, differentiated cells, mature cells, stem cells, such as adherent or suspension primary cells, transfected and non-transfected cell lines, adult cells, embryonic and induced pluripotent stem cells, tissues, tissues inserts, clustered cells, printed cells, an organoid, tissue biopsy, tumor tissue, resected tissue material, an organ explant, an embryonic body, and 3D microtissues, such as muscles and cardiac micro tissues, 3D cultures, such as spheroids and organoids, and combinations thereof.

The invention is further detailed by the accompanying figures and examples, which are exemplary and explanatory of nature and are not limiting the scope of the invention. To the person skilled in the art it may be clear that many variants, being obvious or not, may be conceivable falling within the scope of protection, defined by the present claims.

### EXAMPLES/EXPERIMENTS

The invention although described in detailed explanatory context may be best understood in conjunction with the accompanying examples and figures.

What is claimed is:

1. A micro-fluidic device comprising:
at least one first microchamber having a bottom and at least one wall, wherein the first microchamber comprises an opening such that it is directly accessible from outside,
at least one pillar disposed in the first microchamber, the at least one pillar extending from the bottom upwards for supporting of cell or tissue, wherein the at least one pillar has a height of about 1-5000 μm, and wherein the at least one pillar has a width of about 1-2000 μm, and
at least one first channel in fluidic contact with the at least one microchamber embedded in the bottom, wherein the bottom comprises a porous membrane so as to embody a selective barrier providing fluidic contact between the at least one first channel and the at least one microchamber.

2. The micro-fluidic device according to claim 1, further comprising at least one stimulator.

3. The micro-fluidic device according to claim 1,
wherein the porous membrane comprises, an array of openings,
wherein the array comprises n×m openings, wherein n>10, and m>10,
wherein a density of openings is about 0.001-250/100 μm²,
wherein an average openings area is about 0.05-500 μm², and/or
wherein the at least one pillar is disposed near and/or on top of the at least one opening.

4. The micro-fluidic device according to claim 1, wherein the porous membrane comprises about a 0.05-100 μm thin polymer top layer, the polymer top layer comprising a matrix of holes therein,
a 50-5000 μm thin polymer bottom layer in contact with the polymer top layer,
at least one second micro-channel embedded at least partly in the polymer bottom layer,
wherein the at least one pillar is disposed near and/or on top of the at least one first or second micro-chamber wherein the at least one pillar is disposed near and/or on top of the at least one first or second micro-channel,
wherein the polymer of the polymer film is selected from the group consisting of: glass, silicon oxide, silicon nitride or biocompatible polymers, poly siloxanes, polydimethylsiloxane (PDMS), polyimides, polyurethane, butyl rubber, styrene-ethylene-butylene-styrene (SEBS), polypropylene, polycarbonate, polyester, polypropylene, biodegradable polymers, Biorubber (poly(glycerol sebacate PGS), and poly(1,8-octanediol-co-citrate) (POC), and combinations thereof,
wherein the thin polymer top layer comprises at least one side thereof, at least one micro-feature, an array of x*y oriented microgrooves, wherein a density of microgrooves is about 1-25/100 μm²,
wherein an average groove area is about 0.1-10⁶ μm²,
wherein the at least one micro-feature is aligned with respect to the device,
wherein the polymer layers comprise at least one access, the at least one access providing access to at least one of a metal pad, an IC, a sensor, and a heater,
a rigid substrate forming the microchamber wall, and/or
wherein the least one first microchamber has a shape resembling a biological tissue or organism to be received.

5. The micro-fluidic device according to claim 1, comprising at least one electrode in the bottom, wherein an electrode at one end is in electrical contact with at least one pillar, and wherein the electrode comprises a contact at another end thereof.

6. The micro-fluidic device according to claim 1, wherein the porous membrane and/or the at least one pillar is one selected of a rigid construction and a flexible construction.

7. The micro-fluidic device according to claim 1, wherein the bottom comprises a metal layer disposed between a top layer and bottom layer, wherein the metal layer is patterned, and wherein the metal layer is adapted to detect deformation of the at least one pillar.

8. The micro-fluidic device according to claim 1,
wherein the at least one pillar comprises a cross-section selected from the group consisting of: square, rectangular, oval, elliptic, circular, triangular, multigonal, and combinations thereof,
wherein a cross-section is substantially constant from the bottom upwards or wherein the cross-section gradually increases in area from the bottom upwards,
wherein at least one pillar comprises an optical guider at a top thereof, and/or
wherein at least one pillar is hollow, wherein the hollow part of the pillar is in microfluidic connection with first channel, or with second channel, or with first or second microchamber.

9. The micro-fluidic device according to claim 1, further comprising:
at least one pump embedded in the device, and wherein a wet/humid section and a dry section of the device are physically separated, wherein the dry section comprises electronics.

10. The micro-fluidic device according to claim 1, wherein the bottom comprises a polymer film which is stretchable having a tensile strength of >1 [MPa] (ISO 527), and/or flexible with a Young's modulus of <3 [GPa] (ISO 527), or wherein the polymer film is rigid having a Young's modulus of >10 [GPa] (ISO 527).

11. The micro-fluidic device according to claim 1, further comprising a support, wherein the micro-fluidic device is detachably attached to said support.

12. The micro-fluidic device according to claim 1, comprising at least one living organism or living part thereof, wherein the at least one living organism is selected from the group consisting of: undifferentiated cells, differentiated cells, mature cells, stem cells, adherent or suspension primary cells, endothelial cells, transfected or non-transfected cell lines, adult, embryonic or induced pluripotent stem cells, tissues, tissues inserts, 3D microtissues, muscles and cardiac micro tissues, 3D cultures, spheroids, organoids, and combinations thereof.

13. A method using the device according to claims 1, comprising:
seeding the first microchamber with primary or induced pluripotent stem cell skeletal or cardiac muscle cells with or without myoblasts/fibroblasts/endothelial cells to enable growth of a muscle bundle anchored to the pillars; and
seeding the channel with primary, transfected or induced pluripotent stem cell derived endothelial cells to create a 3D perfusable culture and/or to vascularize the bundle through the porous membrane.

14. A method of applying a stimulus to at least one living organism or living part thereof, comprising:
providing at least one micro-fluidic device according to claim 1,
providing at least one living organism or living part thereof each individually comprising at least one cell,
providing at least one stimulus, and
obtaining a test result.

15. The method according to claim 14, wherein the stimulus is a chemical stimulus, a mechanical stimulus, an electrical stimulus, or an optical stimulus, for toxicity testing of drugs and xenobiotics, for efficacy testing, for modeling barrier tissues in vitro, for integrity assessment and drug transport assays, for Drug metabolism studies, for drug pharmacokinetic and toxicokinetic studies, for metabolizing organ and targeting pharmacological organs, for disease modelling, for disease diagnosis, for studying a disease mechanism, for prognosis, for personalized and precise medicine, for doping, for astronauts, for drug interaction, for cell maturation, and for cell differentiation.

16. The method according to claim 14, wherein the at least one living organism or living part thereof is selected from the group consisting of: undifferentiated cells, differentiated cells, mature cells, stem cells, adherent or suspension primary cells, endothelial cells, transfected and non-transfected cell lines, adult cells, embryonic and induced pluripotent stem cells, tissues, tissues inserts, clustered cells, printed cells, an organoid, tissue biopsy, tumor tissue, resected tissue material, an organ explant, an embryonic body, and 3D microtissues, muscles and cardiac micro tissues, 3D cultures, spheroids and organoids, and combinations thereof.

17. The micro-fluidic device according to claim 2, wherein the stimulator is one of the group consisting of: an electrical stimulator, an electrode, a chemical stimulator, an optical stimulator, a mechanical stimulator, a pump, and a tissue monitor.

18. The micro-fluidic device according to claim 4 wherein the at least one micro-feature is selected from the group consisting of: an indentation, a groove, and a topographical structure,

19. The micro-fluidic device according to claim 8, wherein the optical guider is a pointer and wherein optical guiders of opposite pillars point in the same direction, or in an opposite direction, and combinations thereof,

20. The micro-fluidic device according to claim 5, wherein the electrode is incorporated in an insulating material, and wherein the contact is electrically separated from the wall by a further insulating material.

21. The micro-fluidic device according to claim 1 wherein the device is adapted to receive fluid from a pump, the device further comprising one selected from the group consisting of: a valve, a strain gauge, an actuator, a heater, a cooler, a flow sensor, a temperature sensor, a pH sensor, an IC-circuit, an amplifier, an actuator, a hot plate, a micro-electrode array, an ion sensor, a pressure regulator, further microfluidic elements, at least one of a microchip, an integrated sensor, and an output embedded in the bottom of the device and wherein a wet/humid section and a dry section of the device are physically separated, wherein the dry section comprises electronics.
